(19) United States
(12) Patent Application Publication
(10) Pub. No.: US 2025/0372266 A1
(43) Pub. Date: Dec. 4, 2025

(54) METHOD AND SYSTEM FOR PREDICTING AND EVALUATING PERSONALIZED COLD STRESS RISK BASED ON DEEP LEARNING

(71) Applicants: China University of Mining and Technology-Beijing, Beijing (CN); Inner Mongolia Research Institute, China University of Mining and Technology (Beijing), Ordos (CN)

(72) Inventors: Jiansong WU, Beijing (CN); Chuan LI, Beijing (CN); Boyang SUN, Beijing (CN); Guoyong WANG, Ordos (CN)

(73) Assignees: China University of Mining and Technology-Beijing, Beijing (CN); Inner Mongolia Research Institute, China University of Mining and Technology (Beijing), Ordos (CN)

(21) Appl. No.: 19/213,949

(22) Filed: May 20, 2025

(30) Foreign Application Priority Data
Jun. 3, 2024 (CN) 202410709742.5

(51) Int. Cl. G16H 50/30; G06N 3/04; G06N 3/086

(52) U.S. Cl. CPC G16H 50/30; G06N 3/04; G06N 3/086

(57) ABSTRACT

A method and a system for predicting and evaluating personalized cold stress risk based on deep learning are provided. The method includes sequentially performing data cleaning and formatting on received feature parameters of a subject and environmental variables at a location of the subject; according to a prebuilt thermoregulation model, performing a preliminary prediction on a skin temperature of the subject, and determining a target segment based on a result of the preliminary prediction and formatted feature parameters including one or more of metabolic rate, set-point temperature, or heat capacity; based on a deep learning algorithm, iteratively adjusting the key parameter for the target segment according to the formatted data; and according to the thermoregulation model with adjusted key parameters, performing a secondary prediction on the skin temperature, determining a cold stress risk of the subject based on the result of secondary prediction, and feeding it back to the subject.

### CROSS-REFERENCE TO RELATED DISCLOSURES

This application claims priority to Chinese Patent Application No. 202410709742.5, filed on Jun. 3, 2024, which is hereby incorporated by reference in its entirety.

### TECHNICAL FIELD

The present disclosure relates to the technical field of cold stress risk prediction, and particularly to a method and a system for predicting and evaluating a personalized cold stress risk based on deep learning, a non-transitory computer-readable storage medium, and an electronic device.

### BACKGROUND

As the duration, frequency, and intensity of cold weather phenomena-such as freezing temperatures, snowstorms, and ice hazards, continue to increase, emergency responders, rescue workers, and specialized operators inevitably face extreme low-temperature and freezing conditions, which not only reduces work efficiency and quality but also poses risks of localized or systemic injuries, and may even lead to fatalities.

Thermoregulation models, serving as effective tools for predicting the physiological responses of subjects in extreme environments, are widely used to evaluate thermal stress risks and cold stress risks. However, most current thermoregulation models are derived from Stolwijk model, which utilizes the basic data, such as set-point temperatures, distribution of metabolic rate coefficients for different divisions and segments of each subject, and somatic heat capacity values for each segment that are all at the population level. These thermoregulation models fail to process, analyze, and predict difference data between individuals, resulting in prediction results that lack validity and specificity for different individuals.

In recent years, some analysis and prediction models that incorporate personalized factors such as age, gender, height, and physiological characteristics have, to some extent, improve the accuracy of analysis and prediction at the individual level. However, due to the significant differences between individuals, especially in extremely cold environments where the difference between individuals in trunk, limbs, and other parts are even greater, these analysis and prediction models cannot achieve accurate prediction at the individual level.

Therefore, there is an urgent need to provide a technical solution that gets over the shortcomings in the related art.

### SUMMARY

The present disclosure provides a method for predicting and evaluating a personalized cold stress risk based on deep learning, which can solve or mitigate the problems present in the aforementioned related art.

The present disclosure provides the following technical solutions.

Embodiments of the present disclosure provide a method for predicting and evaluating a personalized cold stress risk based on deep learning. The method includes:
step S101: sequentially performing, by a cloud, data cleaning and formatting on feature parameters of a subject received by the cloud and environmental variables at a location of the subject that are received by the cloud;
step S102: performing, based on a thermoregulation model that is prebuilt, a preliminary prediction on a skin temperature of the subject, and determining, based on an evaluation of an error between a result of the preliminary prediction and the formatted feature parameters, a segment with an average error greater than 275.15 K as a target segment for which at least one key parameter of the thermoregulation model needs to be adjusted, the at least one key parameter including at least one of a metabolic rate, a set-point temperature, or a heat capacity, and the thermoregulation model being:

$$\{ \begin{matrix}{C}_{i , j} \frac{{dT}_{i , j}}{dt} = {Q}_{i , j} - {B}_{i , j} + {D}_{i , j - 1} - {D}_{i , j} - Re {s}_{i . j} - ( {Rad}_{i , 4} + {Con}_{i , 4} + {Eva}_{i , 4} ) \\ {Q}_{i , j} = {M}_{basal} + {M}_{sh} + W\end{matrix}$$

where i denotes a serial number of one segment of a plurality of segments obtained by dividing the subject, and i={1,2, . . . , 22},
j denotes a serial number of one layer of a plurality of layers of each segment of the plurality of segments, and j={1, 2, 3, 4},
C_{i,j} denotes a heat capacity of a j-th layer of the plurality of layers of an i-th segment of the plurality of segments and is in a unit of J/K,
T_{i,j} denotes a temperature of the j-th layer of the i-th segment and is in a unit of ° C.,
t denotes a duration for which the subject is exposed to a cold environment and is in a unit of second,
Q_{i,j} denotes a heat generation of the j-th layer of the i-th segment and is in a unit of W,
B_{i,j} denotes a blood heat exchange of the j-th layer of the i-th segment and is in a unit of W,
D_{i,j-1} and D_{i,j} denote a conductive heat exchange between a (j−1)-th layer and another layer of the plurality of layers of the i-th segment and a conductive heat exchange between the j-th layer and the another layer of the i-th segment, respectively, and each are in a unit of W,
Re s_{i,j} denotes a respiratory heat exchange of the j-th layer of the i-th segment and is in a unit of W,
Rad_{i,4}, Con_{i,4}, and Eva_{i,4} denote a radiant heat exchange between the i-th segment of the subject and ambient environment, a convective heat exchange between the i-th segment of the subject and the ambient environment, and an evaporative heat exchange between the i-th segment of the subject and the ambient environment, respectively, and each are in a unit of W, and
M_{basal}, Mₛₕ, and W denote a basal metabolic rate, a shivering heat generation rate, and a work heat rate, respectively, and each are in a unit of W;
step S103: iteratively adjusting, based on a deep learning algorithm, the at least one key parameter for the target segment according to the formatted feature parameters and the formatted environmental variables; and
step S104: performing, based on the thermoregulation model with the adjusted at least one key parameter, a secondary prediction on the skin temperature of the subject, determining a cold stress risk of the subject based on a result of the secondary prediction, and feeding the cold stress risk back to the subject, where a wind chill warning is issued to the subject in response to that a wind chill temperature calculated according the environmental variables at the location of the subject is lower than a preset wind chill temperature, and a frostbite risk warning is issued to the subject in response to that the skin temperature of the subject obtained by the secondary prediction with the thermoregulation model after the second prediction is lower than or equal to a preset frostbite risk index.

In some embodiments, at step S101, the performing data cleaning includes subsequently performing outlier detection, missing data handling, and noise filtering on the feature parameters and the environmental variables, and the performing formatting includes subsequently performing standardization, feature engineering, and data reconstruction on the feature parameters and the environmental variables obtained after the data cleaning.

In some embodiments, at step S103, based on a genetic algorithm, iterative personalized adjustment is performed, according to a preset priority, on the at least one key parameter for the target segment that needs to be adjusted, until an error between the result of the preliminary prediction of the thermoregulation model and the formatted feature parameters is smaller than a preset temperature threshold.

In some embodiments, at step S104, the wind chill temperature t_{wc} is calculated according to the formatted environmental variables at the location of the subject with a formula:

$${t}_{wc} = 13.12 + 0.6215 \times {t}_{a} - 11.37 \times {v}_{10}^{0.16} + 0.3965 \times {t}_{a} \times {v}_{10}^{0.16} ,$$

where tₐ denotes an air temperature, v₁₀ denotes a wind speed at the location of the subject, and v₁₀>1.34 m/s.

Embodiments of the present disclosure also provide a method for predicting and evaluating a personalized cold stress risk based on deep learning. The method includes: acquiring, by a configured wearable device, feature parameters of a subject and environmental variables; sending, by the configured wearable device, the feature parameters and the environmental variables to a cloud; and receiving, by the configured wearable device, a fed back of a cold stress risk from the cloud, where the cloud is configured to perform, according to the feature parameters and the environmental variables, personalized adjustment on at least one key parameter of a thermoregulation model that is prebuilt, and is configured to determine the cold stress risk of the subject based on the thermoregulation model with the adjusted at least one key parameter.

Embodiments of the present disclosure also provide a system for predicting and evaluating a personalized cold stress risk based on deep learning. The system includes a preprocessing circuit, a target segment determining circuit, a parameter adjusting circuit, and a predicting and feeding-back circuit. The preprocessing circuit is configured to sequentially perform, in a cloud, data cleaning and formatting on feature parameters of a subject that are received by the cloud and environmental variables at a location of the subject that are received by the cloud. The target segment determining circuit is configured to perform, based on a thermoregulation model that is prebuilt, a preliminary prediction on a skin temperature of the subject, and is configured to determine, based on an evaluation of an error between a result of the preliminary prediction and the formatted feature parameters, a segment with an average error greater than 275.15 K as a target segment for which at least one key parameter of the thermoregulation model needs to be adjusted. The at least one key parameter includes at least one of a metabolic rate, a set-point temperature, or a heat capacity. The thermoregulation model is:

$$\{ \begin{matrix}{C}_{i , j} \frac{{dT}_{i , j}}{dt} = {Q}_{i , j} - {B}_{i , j} + {D}_{i , j - 1} - {D}_{i , j} - Re {s}_{i . j} - ( {Rad}_{i , 4} + {Con}_{i , 4} + {Eva}_{i , 4} ) \\ {Q}_{i , j} = {M}_{basal} + {M}_{sh} + W\end{matrix} ,$$

where i denotes a serial number of one segment of a plurality of segments obtained by dividing the subject, and i={1,2, . . . , 22},
j denotes a serial number of one layer of a plurality of layers of each segment of the plurality of segments, and j={1, 2, 3, 4},
C_{i,j} denotes a heat capacity of a j-th layer of the plurality of layers of an i-th segment of the plurality of segments and is in a unit of J/K,
T_{i,j} denotes a temperature of the j-th layer of the i-th segment and is in a unit of ° C., t denotes a duration for which the subject is exposed to a cold environment and is in a unit of second,
Q_{i,j} denotes a heat generation of the j-th layer of the i-th segment and is in a unit of W,
B_{i,j} denotes a blood heat exchange of the j-th layer of the i-th segment and is in a unit of W,
D_{i,j-1} and D_{i,j} denote a conductive heat exchange between a j−1-th layer and another layer of the plurality of layers of the i-th segment and a conductive heat exchange between the j-th layer and the another layer of the i-th segment, respectively, and each are in a unit of W,
Re s_{i,j} denotes a respiratory heat exchange of the j-th layer of the i-th segment and is in a unit of W,
Rad_{i,4}, Con_{i,4}, and Eva_{i,4} denote a radiant heat exchange between the i-th segment of the subject and ambient environment, a convective heat exchange between the i-th segment of the subject and the ambient environment, and an evaporative heat exchange between the i-th segment of the subject and the ambient environment, respectively, and each are in a unit of W, and
M_{basal}, Mₛₕ, and W denote a basal metabolic rate, a shivering heat generation rate, and a work heat rate, respectively, and each are in a unit of W. The parameter adjusting circuit is configured to iteratively adjust, based on a deep learning algorithm, the at least one key parameter for the target segment according to the formatted feature parameters and the formatted environmental variables. The predicting and feeding-back circuit is configured to, perform, based on the thermoregulation model with the adjusted at least one key parameter, a secondary prediction on the skin temperature of the subject, is configured to determine a cold stress risk of the subject based on a result of the secondary prediction, and is configured to feed the cold stress risk back to the subject. A wind chill warning is issued to the subject in response to that a wind chill temperature calculated according the environmental variables at the location of the subject is lower than a preset wind chill temperature, and a frostbite risk warning is issued to the subject in response to that the skin temperature of the subject obtained by the secondary prediction with the thermoregulation model after the second prediction is lower than or equal to a preset frostbite risk index.

Embodiments of the present disclosure also provide a non-transitory computer-readable storage medium storing a computer program, and the computer program performs any one method for predicting and evaluating the personalized cold stress risk based on the deep learning in the above.

Embodiments of the present disclosure also provide an electronic device including a memory and a processor. A program is stored on the memory and executable by the processor. The program, when executed by the processor, causes the processor to perform any one method for predicting and evaluating the personalized cold stress risk based on the deep learning in the above.

### DESCRIPTION OF THE DRAWINGS

The accompanying drawings, which is a part of the present disclosure, are used to provide a further understanding of the present disclosure. The exemplary embodiments of the present disclosure and their descriptions are intended to describe the present disclosure and do not impose any improper limitation on the present disclosure.

FIG. 1 is a flowchart of a method for predicting and evaluating a personalized cold stress risk based on deep learning according to some embodiments of the present disclosure;

FIG. 2 is a schematic diagram of a system for predicting and evaluating a personalized cold stress risk based on deep learning according to some embodiments of the present disclosure;

FIG. 3 is a schematic diagram of an electronic device according to some embodiments of the present disclosure; and

FIG. 4 is a hardware structure of an electronic device according to some embodiments of the present disclosure.

### DETAILED DESCRIPTION

The present disclosure will be described below in detail with reference to the accompanying drawings and in conjunction with the embodiments. Each example is provided to describe the present disclosure, rather than limiting the present disclosure. In fact, it is clear to those skilled in the art that various modifications and variations can be made in the present disclosure without departing from the scope of the present disclosure. For instance, features illustrated or described as part of one embodiment can be applied to another embodiment to obtain yet another embodiment. Thus, it is expected that the present disclosure covers such modifications and variants falling into the scope of the appended claims and their equivalents.

In the related art, thermoregulation models that incorporate personalized factors can predict the skin temperature at individual level more accurately to some extent. However, in extreme environments, these models cannot accurately and effectively provide prediction at individual level due to the influence of differences between different individuals.

Based on the above, the present disclosure proposes a method for predicting and evaluating a personalized cold stress risk based on deep learning. With feature parameters (physiological data) of the subject, environmental variables at the location of the subject, and so on, the method performs personalized adjustment to at least one key parameter of the thermoregulation model with a deep learning algorithm to generate a cold stress risk evaluation model suitable to the individual level, which provides a refined individual-level formulation for outdoor work organization strategies and individual protection strategies, thereby effectively preventing personnel from suffering cold injuries to improving outdoor work efficiency.

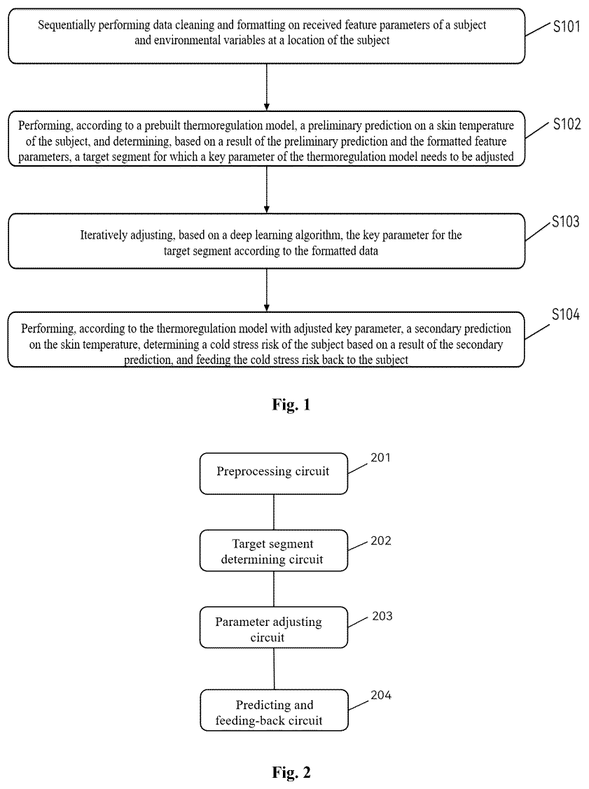

As shown in FIG. 1, the method for predicting and evaluating the personalized cold stress risk based on the deep learning includes steps S101, S102, S103, and S104.

At step S101, data cleaning and formatting are sequentially performed on received feature parameters of a subject and received environmental variables at a location of the subject.

In the present disclosure, the feature parameters of the subject are mainly the physiological parameters of the subject, which are collected by a wearable device of the subject. For example, a heart rate and wrist skin temperature of the subject are detected in real-time through a smart wristwatch worn by the subject, and the skin temperatures at various body parts of the subject is monitored in real-time by skin temperature sensors.

The heart rate monitoring is performed via a heart rate sensor within the smart wristwatch with photoplethysmography technology, and the wrist skin temperature is monitored through a temperature sensor in contact with the skin within the smart wristwatch. The skin temperature sensors used to monitor the skin temperatures at various body parts of the subject may be intelligent fibers attached to different skin parts of the subject or embedded in the clothing worn by the subject.

The environmental variables at the location of the subject are collected in real-time through a portable monitoring device integrating temperature sensor, a humidity sensor, and a wind speed sensor and carried by the subject, and the environmental variables are collected in real-time and transferred via a built-in power supply and a wireless transmission. The portable monitoring device may adopt a portable weather monitor, a multi-functional mobile environmental monitor, a portable weather station, etc.

After the feature parameters of the subject and the environmental variables at the location of the subject are transferred to the cloud, the cloud performs data cleaning and formatting the received physiological parameters (i.e., feature parameters) of the subject and environmental variables. For example, a cloud server first stores the received data in a database, and then sequentially performs outlier detection, missing data handling, and noise filtering on the feature parameters and the environmental variables. Finally, the cloud server standardization, feature engineering, and data reconstruction are performed on the feature parameters and environmental variables that are obtained after the data cleaning, and the data each are formatted in a standardized format.

The cloud server stores the received data in a database, and then sequentially performs outlier detection, missing data handling, and noise filtering on the feature parameters and the environmental variables. When performing outlier detection, first, for the skin temperature and the heart rate, an abnormal range is defined with reference to medical standards, while for the environmental variables, a normal range can be determined according to history data. Next, an outlier for each parameter is detected with statistical methods (such as box plots). Then, the outlier is evaluated to determine whether to delete or retain the outlier. An outlier caused by an error is deleted, and an outlier reflecting an important and atypical condition is retained.

When performing the missing data handling, a missing mode is first identified, for example, the device having a problem if a sensor frequently losing data under a specific condition; a handling strategy is selected based on a data type and an analysis object, for example, for key parameters (such as heart rate), an interpolation method is selected to fill missing data, while for environmental variables, time series analysis methods may be applied.

During a process of performing noise filtering, noise is first identified by detecting a short-term fluctuation that is significantly deviate from an average level; then, the identified data is processed, for example, for data of the skin temperature and the heart rate, the data are smoothed by utilizing moving average or median filters.

Finally, standardization and data reconstruction are sequentially performed on the feature parameters and the environmental variables after the data cleaning is performed, and the data is formatted in the standardized format to ensure that the data is stored in a format suitable for analysis.

At step S102, according to a prebuilt thermoregulation model, a preliminary prediction is performed on a skin temperature of the subject, and a target segment for which at least one key parameter of the thermoregulation model needs to be adjusted is determined according to a result of the preliminary prediction and the formatted feature parameters.

In the present disclosure, the human body is divided into a plurality of segments according to the physiological structure of the human body, and a thermoregulation model of the human body is established. For example, the human body is divided into 22 segments, i.e., head, face, right upper arm, left upper arm, right forearm, left forearm, right hand, left hand, left fingers, right fingers, chest, right shoulder, left shoulder, abdomen, right buttock, left buttock, right thigh, left thigh, right calf, left calf, right foot, left foot. Each segment is divided into a skin layer, a muscle layer, a fat layer, and a core layer. The established thermoregulation model for each segment and layer of the human body is as follows:

$$\{ \begin{matrix}{C}_{i , j} \frac{{dT}_{i , j}}{dt} = {Q}_{i , j} - {B}_{i , j} + {D}_{i , j - 1} - {D}_{i , j} - Re {s}_{i . j} - ( {Rad}_{i , 4} + {Con}_{i , 4} + {Eva}_{i , 4} ) \\ {Q}_{i , j} = {M}_{basal} + {M}_{sh} + W\end{matrix} ,$$

where i denotes a serial number of a divided segment, and i={1,2, . . . , 22}; j denotes a serial number of a layer in each segment, and j={1, 2, 3, 4}; C_{i,j} denotes a heat capacity of a j-th layer of an i-th segment and is in a unit of J/K; T_{i,j} denotes a temperature of the j-th layer of the i-th segment and is in a unit of ° C.; t denotes a duration for which the subject is exposed to a cold environment and is in a unit of second; Q_{i,j} denotes a heat generation of the j-th layer of the i-th segment, is in a unit of W, and includes a basal metabolic rate M_{basal}, a shivering heat generation rate Mₛₕ, and a work heat rate W, which are each in a unit of W; B_{i,j} denotes a blood heat exchange of the j-th layer of the i-th segment and is in a unit of W; D_{i,j-1} and D_{i,j} denote a conductive heat exchange between a (j−1)-th layer and another layer of the plurality of layers of the i-th segment and a conductive heat exchange between the j-th layer and the another layer of the i-th segment, respectively, and each are in a unit of W; Re s_{i,j} denotes a respiratory heat exchange of the j-th layer of the i-th segment and is in a unit of W; and Rad_{i,4}, Con_{i,4}, and Eva_{i,4} denote a radiant heat exchange between the i-th segment of the subject and ambient environment, a convective heat exchange between the i-th segment of the subject and the ambient environment, and an evaporative heat exchange between the i-th segment of the subject and the ambient environment, respectively, and each are in a unit of W.

In the present disclosure, the preliminary prediction is performed on the skin temperature of the subject based on the prebuilt thermoregulation model, and based on evaluation of the error between the result of the preliminary prediction and the actual monitored values (the formatted feature parameters), the target segment for which at least one key parameter of the thermoregulation model needs to be adjusted is determined. For example, the preliminary prediction is performed on original parameters (i.e., initial values) of the thermoregulation model that are shown in Table 1, and the predicted values are compared with the actual monitored values to recognize the segment whose average error is greater than 2° C. This segment is taken as the target segment for which the parameter needs to be adjusted and optimized, and one or more of a metabolic rate (a basal metabolic rate M_{basal}), a set-point temperature (T_{i,j}), or a heat capacity (C_{i,j}) for the target segment are adjusted and optimized. Table 1 is as follows:

TABLE 1
Original parameters of the thermoregulation model

| | Initial |
|---|---|
| Parameter | value |
| Basal metabolic rate of muscle layer: Mmu (W/m³) | 510 |
| Basal metabolic rate of fat layer: Mfa (W/m³) | 62 |
| Basal metabolic rate of skin layer: Msk (W/m³) | 320 |
| Thermal conductivity of muscle layer: Kmu (W · m⁻¹ · K⁻¹) | 0.42 |
| Thermal conductivity of fat layer: Kfa (W · m⁻¹ · K⁻¹) | 0.25 |
| Thermal conductivity of skin layer: Ksk (W · m⁻¹ · K⁻¹) | 0.35 |
| Density of muscle layer: pmu (kg/m³) | 1048 |
| Density of fat layer: pfa (kg/m³) | 865 |
| Density of skin layer: psk (kg/m³) | 1070 |
| Heat capacity of core layer: Cbr (J · kg⁻¹ · K⁻¹) | 3750 |
| Heat capacity of fat layer: Cfa (J · kg⁻¹ · K⁻¹) | 2100 |
| Heat capacity of skin layer: Csk (J · kg⁻¹ · K⁻¹) | 3550 |

At step S103, based on a deep learning algorithm, iteratively adjusting is performed on the key parameter for the target segment according to the formatted data.

In the present disclosure, the segment with an average error greater than 2° C. (i.e., 275.15 K) is selected, and adjustment and optimization are performed on the key parameter for the target segment. For example, based on a genetic algorithm, iterative personalized adjustment is performed, according to a preset priority, on one or more of the metabolic rate (a basal metabolic rate M_{basal}), the set-point temperature (T_{i,j}), or the heat capacity (C_{i,j}) for the target segment that need to be adjusted, until the error between the prediction result of the thermoregulation model and the actual monitored values is smaller than a preset temperature threshold.

In the present disclosure, during the adjustment to the key parameter with the genetic algorithm, the genetic algorithm population includes 200 individuals each having 12 genes that represents active and passive parameters in the human thermoregulation model. These parameters are as shown in Table 2 below.

TABLE 2
Active and passive parameters in the thermoregulation model

| | Initial | Minimum | Maximum |
|---|---|---|---|
| Parameter | value | value | value |
| Mmu (W/m³) | 510 | 385 | 1023 |
| Mfa (W/m³) | 62 | 40 | 108 |
| Msk (W/m³) | 320 | 257 | 684 |
| Kmu (W · m⁻¹ · K⁻¹) | 0.42 | 0.19 | 0.64 |
| Kfa (W · m⁻¹ · K⁻¹) | 0.25 | 0.14 | 0.50 |
| Ksk (W · m⁻¹ · K⁻¹) | 0.35 | 0.16 | 0.96 |
| pmu (kg/m³) | 1048 | 1040 | 1090 |
| pfa (kg/m³) | 865 | 850 | 920 |
| psk (kg/m³) | 1070 | 1000 | 1109 |
| Cbr (J · kg⁻¹ · K⁻¹) | 3750 | 3583 | 3850 |
| Cfa (J · kg⁻¹ · K⁻¹) | 2100 | 1900 | 3800 |
| Csk (J · kg⁻¹ · K⁻¹) | 3550 | 3391 | 3800 |

During the adjustment and optimization process of the key parameter for the target segment, an objective function MAE adopted is as follows:

$$MAE = \frac{1}{N} {\sum }_{i = 1}^{N} {( {predicted}_{i} - {tested}_{i} )}^{2} ,$$

where predicted; denotes a prediction result of the thermoregulation model for the i-th segment of the subject, tested; denotes an actual monitored value (feature parameter) of the i-th segment of the subject, N denotes a number of the segments of the subject, and N=22.

In some embodiments, when adjusting the key parameters with the genetic algorithm, the population is first initialized to define individuals. Each individual is a parameter set of parameters including some values, the parameters includes the basal metabolic rate M_{basal} and the heat capacity (C_{i,j}). Then, a group of individuals is then randomly generated as the initial population, and the parameter set (the basal metabolic rate M_{basal} and the heat capacity (C_{i,j})) of each individual is randomly selected.

Subsequently, based on the objective function MAE, the squared differences between the predicted values and the actual values for all segments are summed and averaged to evaluate a performance of each individual, and based on fitness of the individuals, some individuals with better performance are selected from the current population and are taken as “parents” for next generation in the genetic algorithm.

Some subsets of the basal metabolic rate M_{basal} and the heat capacity (C_{i,j}) are selected from the “parent” individuals, and are exchanged to produce children. Subsequently, some parameters in the individuals are randomly modified, for example, increasing or reducing the basal metabolic rate M_{basal} or adjusting the value of the heat capacity (C_{i,j}), to introduce new genetic mutation. A new generation population is thus generated after selection, crossover, and mutation. The above steps are repeated until the number of iterations or the fitness threshold is reached. Thereafter, the individual with a highest fitness is selected, and the basal metabolic rate M_{basal} and the heat capacity (C_{i,j}) corresponding to this individual are taken as the adjusted and optimized key parameters. The adjusted and optimized key parameters are then applied to the thermoregulation model, to minimize the objective function MAE.

At step S104, based on the thermoregulation model that is adjusted with the key parameters, a secondary prediction is performed on the skin temperature of the subject, a cold stress risk of the subject is determined based on a result of the secondary prediction and is feed back to the subject.

In some embodiments, the environmental variables at the location of the subject are acquired from an environment monitoring instrument or weather forecast to calculate a wind chill temperature. When the wind chill temperature is lower than a preset wind chill temperature, a wind chill warning is issued to the subject. The wind chill temperature t_{wc} is calculated according to a formula:

$${t}_{wc} = 13.12 + 0.6215 \times {t}_{a} - 11.37 \times {v}_{10}^{0.16} + 0.3965 \times {t}_{a} \times {v}_{10}^{0.16} ,$$

where tₐ denotes an air temperature, v₁₀ denotes a wind speed at the location of the subject, and v₁₀>1.34 m/s.

With a risk of the subject being classified into four risk levels based on the wind chill temperature, the wind chill warning is issued to the subject, which is shown in the following Table 3.

TABLE 3
Wind chill temperature evaluation risk classification

| | Preset wind chill | |
|---|---|---|
| Risk level | temperature (° C.) | Impact |
| 1 | −10 to −24 | Cold and uncomfortable |
| 2 | −25 to −34 | Very cold, possible risk |
| | | of skin frostbite |
| 3 | −35 to −59 | Severe cold, exposed skin |
| | | may get frostbite within |
| | | 10 minutes |
| 4 | ≤−60 | Extreme cold, exposed |
| | | skin may get frostbite |
| | | within 2 minutes |

In the present disclosure, local frostbite is divided into freezing frostbite and non-freezing frostbite. When the local skin temperature is between [0° C., 10° C.] for a long time, the local skin suffers from non-freezing frostbite, potentially causing symptoms such as chilblains and immersion foot. When the local skin temperature is below −2.5° C., a local skin tissue suffers from freezing frostbite. Therefore, when a skin temperature of the subject predicted by the thermoregulation model is lower than the preset frostbite risk index, a frostbite risk warning is issued to the subject. In some embodiments, a cold exposure risk is classified into several levels based on the skin temperature to issue the frostbite risk warning to the subject. The classified cold exposure risk levels are shown in Table 4.

TABLE 4
Classified cold exposure risk level

| Skin | | |
|---|---|---|
| temperature | | |
| (° C.) | Cold exposure risk | Impact |
| <−0.25 | Extremely high | Freezing frostbite |
| −0.25 to 0 | High | Non-freezing frostbite |
| 0 to 5 | Moderate | Non-freezing frostbite |
| 5 to 10 | Low | Non-freezing frostbite |
| 10 to 15 | Relatively low | Pain sensation |
| 15 to 20 | None | Cold and uncomfortable |
| >20 | None | No cold exposure risk |

In the present disclosure, a wearable device configured for the subject acquires the feature parameters and the environmental variables of the subject, which are then transmitted to the cloud. The cold stress risk is then feedback to the wearable device from the cloud. The cloud performs personalized adjustment on the key parameters in the prebuilt thermoregulation model according to the feature parameters and the environmental variables, and determines the cold stress risk of the subject (i.e., cold risk) according to the thermoregulation model with the adjusted key parameter.

In view of the above, the physiological parameters of the human body and the environmental data are collected by the configured wearable device. Based on the genetic algorithm, the personalized adjustment is made to the thermoregulation model to more accurately. In this way, the skin temperature at various body parts of the subject during outdoor work can be predicted by taking individual differences (e.g., body mass index, age, gender, and health status) into account, so as to issue personalized risk warnings to the subject, thereby better reflecting the actual condition of the subject and providing a scientific and quantitative health risk evaluation for outdoor work. Consequently, appropriate preventive measures, such as suitable rest time, thermal protection, and necessary medical preparations, can be taken to minimize health threats posed by cold environments.

As shown in FIG. 2, some embodiments of the present disclosure provide a system for predicting and evaluating a personalized cold stress risk based on deep learning. The system includes a preprocessing circuit, a target segment determining circuit, a parameter adjusting circuit, and a predicting and feeding-back circuit.

The preprocessing circuit is configured to sequentially perform data cleaning and formatting on received feature parameters of a subject and environmental variables at a location of the subject.

The target segment determining circuit is configured to perform, based on a prebuilt thermoregulation model, a preliminary prediction on a skin temperature of the subject, and is configured to determine, based on an evaluation of an error between a result of the preliminary prediction and the formatted feature parameters, a segment with an average error greater than 2° C. (i.e., 275.15 K) as a target segment for which at least one key parameter of the thermoregulation model needs to be adjusted. The at least one key parameter includes at least one of a metabolic rate, a set-point temperature, or a heat capacity.

The parameter adjusting circuit is configured to, based on a deep learning algorithm, iteratively adjust the at least one key parameter for the target segment according to the formatted data (i.e., the formatted feature parameters and the formatted environmental variables).

The predicting and feeding-back circuit is configured to perform, based on the thermoregulation model with the adjusted at least one key parameter, a secondary prediction on the skin temperature of the subject, is configured to determine a cold stress risk of the subject based on a result of the secondary prediction, and is configured to feed the cold stress risk back to the subject.

The system for predicting and evaluating the personalized cold stress risk based on the deep learning according to the embodiments of the present disclosure can implement the steps and processes of the method for predicting and evaluating the personalized cold stress risk based on the deep learning provided in any one embodiment of the aforementioned embodiments, and can achieve same technical effects, which will not be repeated herein.

The system may be a general-purpose processor including a central processing circuit (CPU), a network processor (NP), and so on. The processor 401 may also be a digital signal processor (DSP), an application specific integrated circuit (ASIC), a field programmable gate array (FPGA) or other programmable logic devices, discrete gates or transistor logic devices, or discrete hardware components. The processor 401 may implement or execute various methods, steps, and logical block diagrams disclosed in the embodiments of the present disclosure. The general-purpose processor may be a microprocessor, any conventional processor or the like.

The circuits may be implemented as hardware, software, firmware, or a com bination thereof. When implemented as hardware, they may be, for example, an elect ronic circuit, an application specific integrated circuit (ASIC), appropriate firmware, a plug-in, a function card, and the like.

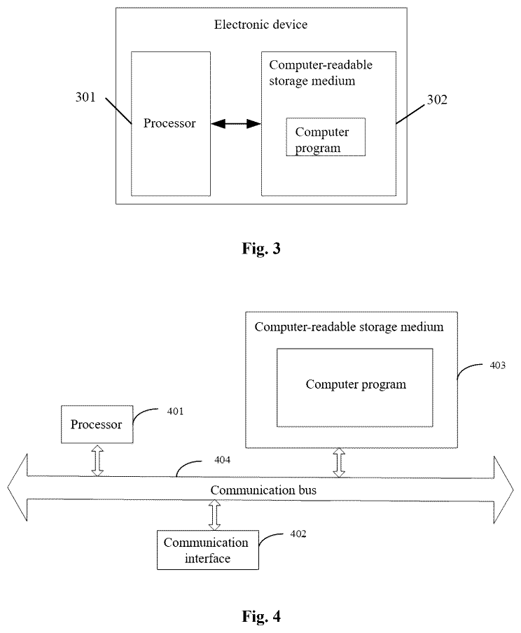

FIG. 3 is a schematic diagram of an electronic device according to some embodiments of the present disclosure. As shown in FIG. 3, the electronic device includes one or more processors 301 and a computer-readable storage medium 302 configured to store one or more programs. The one or more programs, when executed by the one or more processors 301, causes the one or more processors 301 to perform steps: sequentially performing data cleaning and formatting on received feature parameters of a subject and received environmental variables at a location of the subject; performing, according to a prebuilt thermoregulation model, a preliminary prediction on a skin temperature of the subject, and determining, according to a result of the preliminary prediction and the formatted feature parameters, a target segment for which at least one key parameter of the thermoregulation model needs to be adjusted, the at least one key parameter including at least one of a metabolic rate, a set-point temperature, or a heat capacity; iteratively adjusting, based on a deep learning algorithm, the at least one key parameter for the target segment according to the formatted data (i.e., the formatted feature parameters and the formatted environmental variables); and performing, based on the thermoregulation model with the adjusted at least one key parameter, a secondary prediction on the skin temperature of the subject, determining a cold stress risk of the subject based on a result of the secondary prediction, and feeding the cold stress risk back to the subject.

FIG. 4 is a hardware structure of an electronic device according to some embodiments of the present disclosure. As shown in FIG. 4, the hardware structure of the electronic device may include a processor 401, a communication interface 402, a computer-readable medium 403, and a communication bus 404.

The processor 401, the communication interface 402, and the computer-readable storage medium 403 communicate with each other via the communication bus 404.

In some embodiments, the communication interface 402 may be an interface of a communication module, such as an interface of a global system for mobile communications (GSM) module.

The processor 401 can be configured to: sequentially perform data cleaning and formatting on received feature parameters of a subject and received environmental variables at a location of the subject; perform, according to a prebuilt thermoregulation model, a preliminary prediction on a skin temperature of the subject, and determine, according to a result of the preliminary prediction and the formatted feature parameters, a target segment for which at least one key parameter of the thermoregulation model needs to be adjusted, the at least one key parameter including at least one of a metabolic rate, a set-point temperature, or a heat capacity; iteratively adjust, based on a deep learning algorithm, the at least one key parameter for the target segment according to the formatted data (i.e., the formatted feature parameters and the formatted environmental variables); and perform, based on the thermoregulation model with the adjusted at least one key parameter, a secondary prediction on the skin temperature of the subject, determine a cold stress risk of the subject based on a result of the secondary prediction, and feed the cold stress risk back to the subject.

The processor 401 may be a general-purpose processor including a central processing circuit (CPU), a network processor (NP), and so on. The processor 401 may also be a digital signal processor (DSP), an application specific integrated circuit (ASIC), a field programmable gate array (FPGA) or other programmable logic devices, discrete gates or transistor logic devices, or discrete hardware components. The processor 401 may implement or execute various methods, steps, and logical block diagrams disclosed in the embodiments of the present disclosure. The general-purpose processor may be a microprocessor, any conventional processor or the like.

The electronic device in the embodiments of the present disclosure is presented in various forms, including but not limited to a mobile communication device, an ultra-mobile personal computer device, a portable entertainment device, a server, and other electronic device with data communication function.

The mobile communication device is characterized by its mobile communication function and aims to provide voice and data communication. Such terminal includes a smart phone (such as iPhone), a multimedia phone, a functional phone, and a low-end phone.

The ultra-mobile personal computer device belongs to the category of personal computers and has computing and processing functions and generally having character of mobile internet. Such terminal includes a personal digital assistant (PDA), a personal digital assistant (MID) device and an ultra-mobile personal computer (UMPC), e.g., iPad.

The portable entertainment device can display and play multimedia content and includes an audio and video player (such as iPod), a handheld game consoles, an e-book, a smart toy, and a portable car navigation device.

The server provides computing services and includes a processor, a hard disk, a memory, a system bus, and so on. With a similar architecture to a general-purpose computer, the server needs to provide highly reliable services, thus it has a higher processing power, a higher stability, a higher reliability, a higher security, a better scalability, and a better manageability.

According to needs for implementation, the components/steps described in the embodiments of the present disclosure are separated into more components/steps, and two or more components/steps or some operations of the components/steps are also combined into new components/steps to achieve the purpose of the embodiments of the present disclosure.

The above method according to the embodiments of the present disclosure may be implemented in hardware or firmware, or may be implemented as software or computer codes that can be stored in a recording medium (such as a compact disc read-only memory (CD ROM), a random access memory (RAM), a floppy disk, a hard disk or a magneto-optical disk), or may be implemented as computer codes that are downloaded through a network, that are originally stored in a remote recording medium or a non-transitory machine-readable medium, and that will be stored in a local recording medium. Therefore, the method described herein may be processed by a software that is stored in a recording medium that uses a general-purpose computer, a special-purpose processor, or a programmable or dedicated hardware (such as an ASIC or an FPGA). It can be understood that a computer, a processor, a microprocessor controller or a programmable hardware includes a storage component (for example, a RAM, a read-only memory (ROM), or a flash memory) that can store or receive software or a computer code. When accessed and executed by the computer, the processor, or the hardware, the software or the computer code performs the method for predicting and evaluating the personalized cold stress risk based on the deep learning described herein. When the general-purpose computer accesses a code that is used for implementing the method described herein, execution of the code converts the general-purpose computer to a special-purpose computer configured to execute the method herein.

Those skilled in the art may be aware that the examples of the circuits and the method steps described in conjunction with the embodiments disclosed in the description, may be implemented by electronic hardware or a combination of computer software and electronic hardware. Whether these functions are performed by hardware or software depends on the particular applications and design constraint conditions of the technical solutions. Persons skilled in the art may use different methods to implement the described functions for each particular application, but such implementations should not be regarded as beyond the scope of the embodiments of the present disclosure.

The various embodiments in this description are described in a progressive manner. The same or similar contents in various embodiments may be cross-referenced. Each embodiment focuses on describing the differences from other embodiments. In particular, since the device and system embodiments are basically similar to the method embodiments, their descriptions are relatively brief, and relevant parts may refer to those described in the method embodiments.

The device and system embodiments described above are merely exemplary. The circuits described as separate components may or may not be physically separated, and the components referred to as circuits may or may not correspond to physical entities, that is, they may be located in one place, or may be distributed over multiple network units. Some or all of the modules may be selected according to actual needs to achieve the objects of the solutions of the embodiments. Those skilled in the art can understand and implement these embodiments without creative efforts.

The foregoing descriptions are merely exemplary embodiments of the present disclosure and are not intended to limit the scope of the present disclosure. Those skilled in the art may make various modifications and variations. Therefore, all such modifications, equivalent replacement, improvements, etc., without departing from the scope of the embodiments of the present disclosure shall also fall within the scope of the present disclosure.

What is claimed is:

1. A method for predicting and evaluating a personalized cold stress risk based on deep learning, comprising:
step S101: sequentially performing, by a cloud, data cleaning and formatting on feature parameters of a subject that are received by the cloud and environmental variables at a location of the subject that are received by the cloud;
step S102: performing, based on a thermoregulation model that is prebuilt, a preliminary prediction on a skin temperature of the subject, and determining, based on an evaluation of an error between a result of the preliminary prediction and the formatted feature parameters, a segment with an average error greater than 275.15 K as a target segment for which at least one key parameter of the thermoregulation model needs to be adjusted, the at least one key parameter comprising at least one of a metabolic rate, a set-point temperature, or a heat capacity,
wherein the thermoregulation model is:
$$\{ \begin{matrix}{C}_{i , j} \frac{{dT}_{i , j}}{dt} = {Q}_{i , j} - {B}_{i , j} + {D}_{i , j - 1} - {D}_{i , j} - Re {s}_{i . j} - ( {Rad}_{i , 4} + {Con}_{i , 4} + {Eva}_{i , 4} ) \\ {Q}_{i , j} = {M}_{basal} + {M}_{sh} + W\end{matrix} ,$$
where i denotes a serial number of one segment of a plurality of segments obtained by dividing the subject, and i={1,2, . . . , 22},
j denotes a serial number of one layer of a plurality of layers of each segment of the plurality of segments, and j={1, 2, 3, 4},
C_{i,j} denotes a heat capacity of a j-th layer of the plurality of layers of an i-th segment of the plurality of segments and is in a unit of J/K,
T_{i,j} denotes a temperature of the j-th layer of the i-th segment and is in a unit of ° C.,
t denotes a duration for which the subject is exposed to a cold environment and is in a unit of second,
Q_{i,j} denotes a heat generation of the j-th layer of the i-th segment and is in a unit of W,
B_{i,j} denotes a blood heat exchange of the j-th layer of the i-th segment and is in a unit of W,
D_{i,j-1} and D_{i,j} denote a conductive heat exchange between a (j−1)-th layer and another layer of the plurality of layers of the i-th segment and a conductive heat exchange between the j-th layer and the another layer of the i-th segment, respectively, and each are in a unit of W,
Re s_{i,j} denotes a respiratory heat exchange of the j-th layer of the i-th segment and is in a unit of W,
Rad_{i,4}, Con_{i,4}, and Eva_{i,4} denote a radiant heat exchange between the i-th segment of the subject and ambient environment, a convective heat exchange between the i-th segment of the subject and the ambient environment, and an evaporative heat exchange between the i-th segment of the subject and the ambient environment, respectively, and each are in a unit of W, and
M_{basal}, Mₛₕ, and W denote a basal metabolic rate, a shivering heat generation rate, and a work heat rate, respectively, and each are in a unit of W;
step S103: iteratively adjusting, based on a deep learning algorithm, the at least one key parameter for the target segment according to the formatted feature parameters and the formatted environmental variables; and
step S104: performing, based on the thermoregulation model with the adjusted at least one key parameter, a secondary prediction on the skin temperature of the subject, determining a cold stress risk of the subject based on a result of the secondary prediction, and feeding the cold stress risk back to the subject, wherein a wind chill warning is issued to the subject in response to that a wind chill temperature calculated according the environmental variables at the location of the subject is lower than a preset wind chill temperature, and a frostbite risk warning is issued to the subject in response to that the skin temperature of the subject predicted by the thermoregulation model after the second prediction is lower than or equal to a preset frostbite risk index.

2. The method for predicting and evaluating the personalized cold stress risk based on the deep learning as described in claim 1, wherein at step S101, said performing data cleaning comprises subsequently performing outlier detection, missing data handling, and noise filtering on the feature parameters and the environmental variables, and said performing formatting comprises subsequently performing standardization, feature engineering, and data reconstruction on the feature parameters and the environmental variables obtained after the data cleaning.

3. The method for predicting and evaluating the personalized cold stress risk based on the deep learning as described in claim 1, wherein at step S103, based on a genetic algorithm, iterative personalized adjustment is performed, according to a preset priority, on the at least one key parameter for the target segment that needs to be adjusted, until an error between a prediction result of the thermoregulation model and the formatted feature parameters is smaller than a preset temperature threshold.

4. The method for predicting and evaluating the personalized cold stress risk based on the deep learning as described in claim 1, wherein at step S104, the wind chill temperature t_{wc} is calculated according to the formatted environmental variables at the location of the subject with a formula:
$${t}_{wc} = 13.12 + 0.6215 \times {t}_{a} - 11.37 \times {v}_{10}^{0.16} + 0.3965 \times {t}_{a} \times {v}_{10}^{0.16} ,$$
where tₐ denotes an air temperature, v₁₀ denotes a wind speed at the location of the subject, and v₁₀>1.34 m/s.

5. A method for predicting and evaluating a personalized cold stress risk based on deep learning, comprising:
acquiring, by a configured wearable device, feature parameters of a subject and environmental variables;
sending, by the configured wearable device, the feature parameters and the environmental variables to a cloud; and
receiving, by the configured wearable device, a fed back of a cold stress risk from the cloud, wherein the cloud is configured to perform, according to the feature parameters and the environmental variables, personalized adjustment on at least one key parameter of a thermoregulation model that is prebuilt, and is configured to determine the cold stress risk of the subject based on the thermoregulation model with the adjusted at least one key parameter.

6. A system for predicting and evaluating a personalized cold stress risk based on deep learning, comprising:
a preprocessing circuit configured to sequentially perform, in a cloud, data cleaning and formatting on feature parameters of a subject that are received by the cloud and environmental variables at a location of the subject that are received by the cloud;
a target segment determining circuit configured to perform, based on a thermoregulation model that is prebuilt, a preliminary prediction on a skin temperature of the subject, and configured to determine, based on an evaluation of an error between a result of the preliminary prediction and the formatted feature parameters, a segment with an average error greater than 275.15 K as a target segment for which at least one key parameter of the thermoregulation model needs to be adjusted, the at least one key parameter comprising at least one of a metabolic rate, a set-point temperature, or a heat capacity,
wherein the thermoregulation model is:
$$\{ \begin{matrix}{C}_{i , j} \frac{{dT}_{i , j}}{dt} = {Q}_{i , j} - {B}_{i , j} + {D}_{i , j - 1} - {D}_{i , j} - Re {s}_{i . j} - ( {Rad}_{i , 4} + {Con}_{i , 4} + {Eva}_{i , 4} ) \\ {Q}_{i , j} = {M}_{basal} + {M}_{sh} + W\end{matrix} ,$$
where i denotes a serial number of one segment of a plurality of segments obtained by dividing the subject, and i={1,2, . . . , 22},
j denotes a serial number of one layer of a plurality of layers of each segment of the plurality of segments, and j={1, 2, 3, 4},
C_{i,j} denotes a heat capacity of a j-th layer of the plurality of layers of an i-th segment of the plurality of segments and is in a unit of J/K,
T_{i,j} denotes a temperature of the j-th layer of the i-th segment and is in a unit of ° C.,
t denotes a duration for which the subject is exposed to a cold environment and is in a unit of second,
Q_{i,j} denotes a heat generation of the j-th layer of the i-th segment and is in a unit of W,
B_{i,j} denotes a blood heat exchange of the j-th layer of the i-th segment and is in a unit of W,
D_{i,j-1} and D_{i,j} denote a conductive heat exchange between a (j−1)-th layer and another layer of the plurality of layers of the i-th segment and a conductive heat exchange between the j-th layer and the another layer of the i-th segment, respectively, and each are in a unit of W,
Re s_{i,j} denotes a respiratory heat exchange of the j-th layer of the i-th segment and is in a unit of W,
Rad_{i,4}, Con_{i,4}, and Eva_{i,4} denote a radiant heat exchange between the i-th segment of the subject and ambient environment, a convective heat exchange between the i-th segment of the subject and the ambient environment, and an evaporative heat exchange between the i-th segment of the subject and the ambient environment, respectively, and each are in a unit of W, and
M_{basal}, Mₛₕ, and W denote a basal metabolic rate, a shivering heat generation rate, and a work heat rate, respectively, and each are in a unit of W;
a parameter adjusting circuit configured to iteratively adjust, based on a deep learning algorithm, the at least one key parameter for the target segment according to the formatted feature parameters and the formatted environmental variables; and
a predicting and feeding-back circuit configured to, perform, based on the thermoregulation model with the adjusted at least one key parameter, a secondary prediction on the skin temperature of the subject, configured to determine a cold stress risk of the subject based on a result of the secondary prediction, and configured to feed the cold stress risk back to the subject, wherein a wind chill warning is issued to the subject in response to that a wind chill temperature calculated according the environmental variables at the location of the subject is lower than a preset wind chill temperature, and a frostbite risk warning is issued to the subject in response to that the skin temperature of the subject obtained by the secondary prediction with the thermoregulation model after the second prediction is lower than or equal to a preset frostbite risk index.

7. A non-transitory computer-readable storage medium, storing a computer program, wherein the computer program performs the method for predicting and evaluating the personalized cold stress risk based on the deep learning as described in claim 1.

8. The non-transitory computer-readable storage medium as described in claim 7, wherein at step S101, said performing data cleaning comprises subsequently performing outlier detection, missing data handling, and noise filtering on the feature parameters and the environmental variables, and said performing formatting comprises subsequently performing standardization, feature engineering, and data reconstruction on the feature parameters and the environmental variables obtained after the data cleaning.

9. The non-transitory computer-readable storage medium as described in claim 7, wherein at step S103, based on a genetic algorithm, iterative personalized adjustment is performed, according to a preset priority, on the at least one key parameter for the target segment that needs to be adjusted, until an error between the result of the preliminary prediction of the thermoregulation model and the formatted feature parameters is smaller than a preset temperature threshold.

10. The non-transitory computer-readable storage medium as described in claim 1, wherein at step S104, the wind chill temperature t_{wc} is calculated according to the formatted environmental variables at the location of the subject with a formula:
$${t}_{wc} = 13.12 + 0.6215 \times {t}_{a} - 11.37 \times {v}_{10}^{0.16} + 0.3965 \times {t}_{a} \times {v}_{10}^{0.16} ,$$
where tₐ denotes an air temperature, v₁₀ denotes a wind speed at the location of the subject, and v₁₀>1.34 m/s.

11. An electronic device, comprising a memory and a processor, wherein a program is stored on the memory and executable by the processor, wherein the program, when executed by the processor, causes the processor to perform the method for predicting and evaluating the personalized cold stress risk based on the deep learning as described in claim 1.

12. The electronic device as described in claim 11, wherein at step S101, said performing data cleaning comprises subsequently performing outlier detection, missing data handling, and noise filtering on the feature parameters and the environmental variables, and said performing formatting comprises subsequently performing standardization, feature engineering, and data reconstruction on the feature parameters and the environmental variables obtained after the data cleaning.

13. The electronic device as described in claim 11, wherein at step S103, based on a genetic algorithm, iterative personalized adjustment is performed, according to a preset priority, on the at least one key parameter for the target segment that needs to be adjusted, until an error between the result of the preliminary prediction of the thermoregulation model and the formatted feature parameters is smaller than a preset temperature threshold.

14. The electronic device as described in claim 11, wherein at step S104, the wind chill temperature t_{wc} is calculated according to the formatted environmental variables at the location of the subject with a formula:
$${t}_{wc} = 13.12 + 0.6215 \times {t}_{a} - 11.37 \times {v}_{10}^{0.16} + 0.3965 \times {t}_{a} \times {v}_{10}^{0.16} ,$$
where tₐ denotes an air temperature, v₁₀ denotes a wind speed at the location of the subject, and v₁₀>1.34 m/s.
